(19) United States
(12) Patent Application Publication
(10) Pub. No.: US 2023/0354862 A1
(43) Pub. Date: Nov. 9, 2023

(54) ANTI-DIABETIC COMPOSITION CONTAINING BEET MIXTURE AS ACTIVE INGREDIENT

(71) Applicant: BAE RYEO INNOVATION CO., LTD., Jeju-si (KR)

(72) Inventor: Dae-sik HAM, Jeju-si (KR)

(21) Appl. No.: 17/927,892

(22) PCT Filed: Jan. 26, 2022
(86) PCT No.: PCT/KR2022/001345

§ 371 Date: Nov. 27, 2022

(30) Foreign Application Priority Data
Jan. 27, 2021 (KR) 10-2021-0011648

(51) Int. Cl. A23L 33/105; A61K 36/21; A61K 36/23; A61K 36/73; A61K 36/81; A61K 36/31; A61P 3/10; A23L 2/02

(52) U.S. Cl. CPC A23L 33/105; A61K 36/21; A61K 36/23; A61K 36/73; A61K 36/81; A61K 36/31; A61P 3/10; A23L 2/02; A61K 2236/31; A61K 2236/19

(57) ABSTRACT

The present invention relates to an anti-diabetic composition comprising a fermented product obtained by low-temperature aging a mixture of beets, carrots and apples. More specifically, an aged product, obtained by low-temperature aging of a mixture obtained by extracting and blending juices of beets, carrots and apples in a weight ratio of 1.5-3.5:1.5-2.5:4-7, has excellent blood sugar-reducing effects, such as concentration-dependent suppression of α-glucosidase and α-amylase activity, blood sugar reduction, reduction of fructosamime, insulin, HOMA-IR and HOMA-β content in the blood, and reduction of the area of β cells in Langerhans islands in pancreatic tissue, and thus can be useful as an anti-diabetic composition.

### TECHNICAL FIELD

The present invention relates to an antidiabetic composition containing a beet mixture, and more particularly, to an antidiabetic composition containing a fermentation product obtained by low-temperature fermentation of a mixture of beets, carrots and apples.

### BACKGROUND ART

Diabetes mellitus is a chronic metabolic disease with high prevalence characterized by hyperglycemia. It is a disease of the endocrine system caused by insufficient insulin secretion from the pancreas, and is classified into insulin-dependent diabetes and non-insulin-dependent diabetes. Insulin-dependent diabetes mellitus is type 1 diabetes caused by an autoimmune disease and is associated with genetic factors and viruses.

Non-insulin-dependent diabetes is type 2 diabetes, accounting for more than 90% of diabetes, and causes impaired glucose metabolism due to insulin deficiency.

Drugs such as insulin secretagogues and carbohydrate absorption inhibitors are used for the treatment of type 2 diabetes, but the long-term use thereof is limited due to various side effects. Therefore, research has been actively conducted on natural products to find diabetic drugs for alleviating or preventing diabetes.

Beet is a plant cultivated in the United States, Europe, and Africa, and is also used medicinally because of its medicinal value. In Korea, beets are cultivated in Jeju Island. Beets are low in calories and rich in minerals and vitamins, and thus are beneficial to health.

In the United States and Europe, beets are made into juices or tablets that are used as health functional foods. It is known that different parts of beets have different biological activities. It is known that the leaves of beets are effective in diuretic, anti-inflammation, alleviation of spleen and liver diseases, and alleviation of paralysis, and the roots thereof have biological activities such as antioxidant, anti-inflammatory, anti-tumor, and immune modulatory activities. These biological activities are known to be due to components such as anthocyanins, betanins, phenols and flavonoids, which are contained in beets.

However, no studies have been reported on the effect of beets on diabetes.

Accordingly, the present inventors have conducted studies to evaluate the antidiabetic effect of beets, and as a result, have found that a fermentation product, obtained by mixing beets, carrots and apples at an appropriate mixing ratio, adjusting the acidity of the mixture, and then naturally fermenting the mixture at low temperature by various microorganisms and yeasts present therein, exhibited inhibitory activities against α-amylase and α-glucosidase, reduced elevated blood glucose levels in diabetic mouse models, alleviated common symptoms of diabetes in the mouse models, and affected diabetes-related factors in the blood in the mouse models, reduced insulin concentration, insulin resistance, and serum insulin secretory capacity, and reduced insulin levels in Langerhans islets, and thus have revealed that the beet mixture may be used as an antidiabetic composition, thereby completing the present invention.

### PRIOR ART DOCUMENTS

### Non-Patent Documents

Naver Knowledge Encyclopedia (https://terms.naver.com/entry.nhn?docId=4368446&cid=42776&categoryId=59916)
Wikipedia (https://namu.wiki/w/%EB%B9%84%ED%8A%B8(%EC%B1%84%EC%86%8C)

### DETAILED DESCRIPTION OF THE INVENTION

### Technical Problem

An object of the present invention is to provide an antidiabetic composition containing a fermented beet mixture.

Another object of the present invention is to provide a method for preparing an antidiabetic composition containing a fermented beet mixture.

### Technical Solution

To achieve the above objects, the present invention provides a food composition for preventing and alleviating diabetes, the food composition containing a fermentation broth obtained by juicing beets, carrots and apples at a weight ratio of 1.5 to 3.5:1.5 to 2.5:4 to 7, mixing the juices together, and fermenting the juice mixture at low temperature.

The present invention also provides a food composition for preventing and alleviating diabetes, the food composition containing a fermentation broth obtained by low-temperature fermentation of a mixture of:
a first fermentation broth obtained by juicing beets, carrots and apples at a weight ratio of 1.5 to 3.5:1.5 to 2.5:4 to 7, mixing the juices together, and fermenting the juice mixture at low temperature; and
a second juice obtained by mixing tomatoes, broccoli, and cabbage at a weight ratio of 0.2 to 0.5:1 to 2:1 to 2, followed by boiling, and juicing the boiled m fixture.

The present invention also provides a method for preparing a food composition for preventing and alleviating diabetes, the method including steps of:
i) juicing beets, carrots and apples at a weight ratio of 1.5 to 3.5:1.5 to 2.5:4 to 7, and mixing the juices together, thereby preparing a mixture; and
ii) fermenting the mixture of step i) at a low temperature of −3° C. to 10° C. for 1 to 3 days, thereby preparing a fermentation broth.

The present invention also provides a method for preparing a food composition for preventing and alleviating diabetes, the method including steps of:
a) preparing the fermentation broth of claim 7;
b) mixing tomatoes, broccoli and cabbage at a weight ratio of 0.2 to 0.5:1 to 2:1 to 2, followed by boiling, and juicing the boiled mixture, thereby preparing a juice;
c) mixing the fermentation broth of step a) and the juice of step b) at a weight ratio of 1 to 2:1, thereby preparing a mixture; and
d) fermenting the mixture of step c) at a low temperature of −3° C. to 10° C. for 1 to 8 hours.

The present invention also provides a method for preventing or alleviating diabetes, the method including a step of administering to a patient a fermentation broth obtained by juicing beets, carrots and apples at a weight ratio of 1.5 to 3.5:1.5 to 2.5:4 to 7, mixing the juices together, and fermenting the juice mixture at low temperature.

The present invention also provides a method for preventing or alleviating diabetes, the method including a step of administering to a patient a fermentation broth obtained by low-temperature fermentation of a mixture of: a first fermentation broth obtained by juicing beets, carrots and apples at a weight ratio of 1.5 to 3.5:1.5 to 2.5:4 to 7, mixing the juices together, and fermenting the juice mixture at low temperature; and a second juice obtained by mixing tomatoes, broccoli, and cabbage at a weight ratio of 0.2 to 0.5:1 to 2:1 to 2, followed by boiling, and juicing the boiled mixture.

The present invention also provides a fermentation broth, obtained by juicing beets, carrots and apples at a weight ratio of 1.5 to 3.5:1.5 to 2.5:4 to 7, mixing the juices together, and fermenting the juice mixture at low temperature, for use in preventing and alleviating diabetes.

The present invention also provides a fermentation broth, obtained by low-temperature fermentation of a mixture of: a first fermentation broth obtained by juicing beets, carrots and apples at a weight ratio of 1.5 to 3.5:1.5 to 2.5:4 to 7, mixing the juices together, and fermenting the juice mixture at low temperature; and a second juice obtained by mixing tomatoes, broccoli, and cabbage at a weight ratio of 0.2 to 0.5:1 to 2:1 to 2, followed by boiling, and juicing the boiled mixture, for use in preventing and alleviating diabetes.

### Advantageous Effects

The beet mixture according to the present invention exhibits excellent blood glucose control effect by inhibiting the activities of α-glucosidase and α-amylase in a concentration-dependent manner, reducing blood glucose levels, reducing serum fructosamine, insulin, HOMA-IR and HOMA-β levels, and reducing the area of Langerhans islet p-cells in pancreatic tissue, indicating that it may be useful as an antidiabetic composition.

Specifically, in the present invention, as a result of measuring the α-glucosidase inhibitory activity of the beet mixture in an in vitro test, it was shown that the beet mixture had an inhibitory activity of 30.58±1.01% at 30 mg/ml, which is the highest concentration of the sample. The α-amylase inhibitory activity of the beet mixture started to increase significantly from a concentration of 10 μg/ml, and 98.23±2.08% at s concentration of 30 mg/ml, which is the highest concentration of the sample, indicating that the beet mixture inhibited α-amylase activity in a concentration-dependent manner. In addition, in OSTT and OATT experiments conducted using SD rats, it was shown that 100 mg/kg (B 100) of the beet mixture tended to reduce blood glucose levels or significantly reduced blood glucose levels, compared to a control. In addition, in a type 2 diabetes model, administration of 50 mg/kg (B 50) the beet mixture reduced elevated blood glucose levels, significantly increased the weight of pancreatic tissue. In hematological analysis, the beet mixture reduced fructosamine, insulin, HOMA-IR and HOMA-β levels compared to a control, and reduced the area of Langerhans Islet β-cells to a normal level.

### DESCRIPTION OF DRAWINGS

FIG. 1 is a graph showing the effect of a beet mixture according to the present invention on the inhibition of α-glucosidase activity.

FIG. 2 is a graph showing the effect of the beet mixture according to the present invention on the inhibition of α-amylase activity.

FIG. 3 is a graph showing the effect of the beet mixture according to the present invention on an oral starch tolerance test.

FIG. 4 is a graph showing the results of analyzing the area under the curve (AUC) after the oral starch tolerance test at different concentrations of the beet mixture according to the present invention.

FIG. 5 is a graph showing the effect of the beet mixture according to the present invention on an oral sucrose tolerance test.

FIG. 6 is a graph showing the results of analyzing the area under the curve (AUC) after the oral sucrose tolerance test at different concentrations of the beet mixture according to the present invention.

FIG. 7 is a graph showing the effect of the beet mixture according to the present invention on changes in weekly body weight in a type 2 diabetes model.

FIG. 8 is a graph showing the effect of the beet mixture according to the present invention on changes in weekly food intake in a type 2 diabetes model.

FIG. 9 is a graph showing the effect of the beet mixture according to the present invention on changes in weekly water intake in a type 2 diabetes model.

FIG. 10 is a graph showing the effect of the beet mixture according to the present invention on changes in weekly blood glucose levels in a type 2 diabetes model.

FIG. 11 is a graph showing the effect of the beet mixture according to the present invention on an oral glucose tolerance test in a type 2 diabetes model.

FIG. 12 is a graph showing the results of analyzing the area under the curve (AUC) after the oral glucose tolerance test at different concentrations of the beet mixture according to the present invention.

FIG. 13 is a graph showing the effect of the beet mixture according to the present invention on HbA1c in a type 2 diabetes model.

FIG. 14 is a graph showing the effect of the beet mixture according to the present invention on liver tissue weight in a type 2 diabetes model.

FIG. 15 is a graph showing the effect of the beet mixture according to the present invention on kidney tissue weight in a type 2 diabetes model.

FIG. 16 is a graph showing the effect of the beet mixture according to the present invention on epididymal fat tissue weight in a type 2 diabetes model.

FIG. 17 is a graph showing the effect of the beet mixture according to the present invention on pancreatic tissue weight in a type 2 diabetes model.

FIG. 18 is a graph showing the effect of the beet mixture according to the present invention on serum fructosamine level in a type 2 diabetes model.

FIG. 19 is a graph showing the effect of the beet mixture according to the present invention on serum total cholesterol level in a type 2 diabetes model.

FIG. 20 is a graph showing the effect of the beet mixture according to the present invention on serum HDL cholesterol level in a type 2 diabetes model.

FIG. 21 is a graph showing the effect of the beet mixture according to the present invention on serum triglyceride level in a type 2 diabetes model.

FIG. 22 is a graph showing the effect of the beet mixture according to the present invention on serum LDL cholesterol level in a type 2 diabetes model.

FIG. 23 is a graph showing the effect of the beet mixture according to the present invention on serum insulin level in a type 2 diabetes model.

FIG. 24 is a graph showing the effect of the beet mixture according to the present invention on HOMA-IR in a type 2 diabetes model.

FIG. 25 is a graph showing the effect of the beet mixture according to the present invention on HOMA-β in a type 2 diabetes model.

FIG. 26 is a graph showing the effect of the beet mixture according to the present invention on the β-cell area in a type 2 diabetes model.

FIG. 27 is a graph showing the effect of the beet mixture according to the present invention on pancreatic immunostaining in a type 2 diabetes model (A: normal, B: control, C: B 10, D: B 30, E: B 50, F: positive, scale bar 100 μm, ×100).

### BEST MODE

Hereinafter, the present invention will be described in detail.

The present invention provides a food composition for preventing and alleviating diabetes, the food composition containing a fermentation broth obtained by juicing beets, carrots and apples at a weight ratio of 1.5 to 3.5:1.5 to 2.5:4 to 7, mixing the juices together, and fermenting the juice mixture at low temperature.

The acidity of the mixture is preferably pH 4 to 5.5.

The fermentation at low temperature is preferably performed at a low temperature of −3° C. to 10° C. for 1 to 3 days.

The fermentation broth is preferably obtained by natural fermentation with various microorganisms and yeast.

The term “food composition” is a term that includes all of health food, health functional food and health supplement food, which may all be used.

For the food composition, the fermented mixture of the present invention may be used alone or together with other foods or food ingredients, and may be appropriately used according to a conventional method. The content of the active ingredient may be appropriately determined according to the purpose of use (prevention, health or hygiene). In general, in the production of food or beverage, the fermented mixture of the present invention is added in an amount of 90 parts by weight or less, preferably 50 parts by weight or less, based on the total weight of food or beverage. However, in the case of long-term intake for health and hygiene purposes or for health care purposes, the above amount may be smaller than the lower limit of the above range, and the active ingredient may also be used in an amount larger than the upper limit of the above range because it has no problem in terms of safety.

There is no particular limitation on the kind of food in the food composition. Examples of foods to which the fermented mixture of the present invention may be added include meat, sausages/hams, breads, chocolates, candies, snacks, confectionery, pizza, ramen, other noodles, gums, dairy products including ice cream, various soups, beverages, teas, drinks, alcoholic beverages, and vitamin complexes, and include all types of food in a conventional sense.

The food may additionally contain various sweetening agents or natural carbohydrates. Examples of the natural carbohydrates include monosaccharides such as glucose and fructose, disaccharides such as maltose and sucrose, polysaccharides such as cyclodextrin, and sugar alcohols such as xylitol, sorbitol, and erythritol. Examples of the sweetening agents include natural flavorings such as thaumatin and stevia extracts, and synthetic flavorings such as saccharin, aspartame, etc.

The food composition may contain various nutrients, vitamins, electrolytes, flavoring agents, colorants, pectic acid and its salt, alginic acid and its salt, organic acids, protective colloidal thickeners, pH adjusters, stabilizers, preservatives, glycerin, alcohols, carbonizing agents that are used in carbonated beverages, etc. The content of these additives in the food composition is not so critical, but is generally in the range of 0.001 to 1 part by weight based on 100 parts by weight of the food composition of the present invention.

The present invention also provides a food composition for preventing and alleviating diabetes, the food composition containing a fermentation broth obtained by low-temperature fermentation of a mixture of:
a first fermentation broth obtained by juicing beets, carrots and apples at a weight ratio of 1.5 to 3.5:1.5 to 2.5:4 to 7, mixing the juices together, and fermenting the juice mixture at low temperature; and
a second juice obtained by mixing tomatoes, broccoli, and cabbage at a weight ratio of 0.2 to 0.5:1 to 2:1 to 2, followed by boiling, and juicing the boiled mixture.

Preferably, the first fermentation broth and the second juice are mixed together at a weight ratio of 1 to 2:1.

The mixture of the first fermentation broth and the second juice is preferably fermented at a low temperature of −3° C. to 10° C. for 1 to 8 hours.

The term “food composition” is a term that includes all of health food, health functional food and health supplement food, which may all be used.

For the food composition, the fermented mixture of the present invention may be used alone or together with other foods or food ingredients, and may be appropriately used according to a conventional method. The content of the active ingredient may be appropriately determined according to the purpose of use (prevention, health or hygiene). In general, in the production of food or beverage, the fermented mixture of the present invention is added in an amount of 90 parts by weight or less, preferably 50 parts by weight or less, based on the total weight of food or beverage. However, in the case of long-term intake for health and hygiene purposes or for health care purposes, the above amount may be smaller than the lower limit of the above range, and the active ingredient may also be used in an amount larger than the upper limit of the above range because it has no problem in terms of safety.

There is no particular limitation on the kind of food in the food composition. Examples of foods to which the fermented mixture of the present invention may be added include meat, sausages/hams, breads, chocolates, candies, snacks, confectionery, pizza, ramen, other noodles, gums, dairy products including ice cream, various soups, beverages, teas, drinks, alcoholic beverages, and vitamin complexes, and include all types of food in a conventional sense.

The food may additionally contain various sweetening agents or natural carbohydrates. Examples of the natural carbohydrates include monosaccharides such as glucose and fructose, disaccharides such as maltose and sucrose, polysaccharides such as cyclodextrin, and sugar alcohols such as xylitol, sorbitol, and erythritol. Examples of the sweetening agents include natural flavorings such as thaumatin and stevia extracts, and synthetic flavorings such as saccharin, aspartame, etc.

The food composition may contain various nutrients, vitamins, electrolytes, flavoring agents, colorants, pectic acid and its salt, alginic acid and its salt, organic acids, protective colloidal thickeners, pH adjusters, stabilizers, preservatives, glycerin, alcohols, carbonizing agents that are used in carbonated beverages, etc. The content of these additives in the food composition is not so critical, but is generally in the range of 0.001 to 1 part by weight based on 100 parts by weight of the food composition of the present invention.

The present invention also provides a method for preparing a food composition for preventing and alleviating diabetes, the method including steps of:
i) juicing beets, carrots and apples at a weight ratio of 1.5 to 3.5:1.5 to 2.5:4 to 7, and mixing the juices together, thereby preparing a mixture; and
ii) fermenting the mixture of step i) at a low temperature of −3° C. to 10° C. for 1 to 3 days, thereby preparing a fermentation broth.

The present invention also provides a method for preparing a food composition for preventing and alleviating diabetes, the method including steps of:
i) preparing the fermentation broth of claim 7;
b) mixing tomatoes, broccoli and cabbage at a weight ratio of 0.2 to 0.5:1 to 2:1 to 2, followed by boiling, and juicing the boiled mixture, thereby preparing a juice;
c) mixing the fermentation broth of step a) and the juice of step b) at a weight ratio of 1 to 2:1, thereby preparing a mixture; and
d) fermenting the mixture of step c) at a low temperature of −3° C. to 10° C. for 1 to 8 hours.

The present invention also provides a method for preventing or alleviating diabetes, the method including a step of administering to a patient a fermentation broth obtained by juicing beets, carrots and apples at a weight ratio of 1.5 to 3.5:1.5 to 2.5:4 to 7, mixing the juices together, and fermenting the juice mixture at low temperature.

The present invention also provides a method for preventing or alleviating diabetes, the method including a step of administering to a patient a fermentation broth obtained by low-temperature fermentation of a mixture of: a first fermentation broth obtained by juicing beets, carrots and apples at a weight ratio of 1.5 to 3.5:1.5 to 2.5:4 to 7, mixing the juices together, and fermenting the juice mixture at low temperature; and a second juice obtained by mixing tomatoes, broccoli, and cabbage at a weight ratio of 0.2 to 0.5:1 to 2:1 to 2, followed by boiling, and juicing the boiled m fixture.

The present invention also provides a fermentation broth, obtained by juicing beets, carrots and apples at a weight ratio of 1.5 to 3.5:1.5 to 2.5:4 to 7, mixing the juices together, and fermenting the juice mixture at low temperature, for use in preventing and alleviating diabetes.

The present invention also provides a fermentation broth, obtained by low-temperature fermentation of a mixture of: a first fermentation broth obtained by juicing beets, carrots and apples at a weight ratio of 1.5 to 3.5:1.5 to 2.5:4 to 7, mixing the juices together, and fermenting the juice mixture at low temperature; and a second juice obtained by mixing tomatoes, broccoli, and cabbage at a weight ratio of 0.2 to 0.5:1 to 2:1 to 2, followed by boiling, and juicing the boiled mixture, for use in preventing and alleviating diabetes.

Hereinafter, the present invention will be described in detail with reference to Examples and Experimental Examples.

However, the following Examples and Experimental Examples are only for illustrating of the present invention, and the scope of the present invention is not limited to the following Examples and Experimental Examples.

### <Example 1> Preparation of Beet Mixture

<1-1> Step 1

Beets and carrots, produced in Jeju Island, Korean, apples produced in Korea were used. Beets and carrots were used after peeling, and apples were used after seed removal. Beets, carrots and apples were juiced at a weight ratio of 1.5 to 3.5:1.5 to 2.5:4 to 7, and then mixed together. At this time, the mixing ratio was adjusted to match an acidity of pH 4 to 5.5. The mixture was fermented at a low temperature of −3° C. to 10° C. for one day or more.

<1-2> Step 2

Tomatoes, broccoli and cabbage were boiled at 100° C. for 5 to 15 minutes at a weight ratio of 0.2 to 0.5:1 to 2:1 to 2. At this time, the ratio between the materials may vary depending on the fructose content of each material. For example, carrots contain twice as much fructose as glucose. On the other hand, broccoli is low in fructose. The main key is to increase the content of fructose and increase the proportion of sucrose, enhancing digestion and absorption. Then, the boiled materials were juiced. The fermentation broth of step 1 was mixed with the juice of step 2 at a ratio of 1 to 2:1. Then, the mixture was fermented at a low temperature of −3° C. to 10° C. for 1 to 8 hours. At this time, it is important to ensure sufficient space in consideration of oxygen circulation, and otherwise, ventilation shall be provided.

### <Experimental Example 1> In Vitro Test

<1-1> Alpha-Glucosidase Assay

α-Glucosidase inhibition assay was performed using the method of Tibbot and Skadsen (1996). As an enzyme, α-glucosidase (Sigma, USA) obtained from yeast was used, and as a substrate, p-nitrophenyl-α-D-glucopyranoside (Sigma, USA) was used. α-Glucosidase was dissolved in 100 mM phosphate buffer (pH 7.0) containing 0.2% BSA and 0.02% NaN₃ at a concentration of 0.7 units, thus preparing an enzyme solution, and p-nitrophenyl-α-D-glucopyranoside was dissolved in 100 mM phosphate buffer (pH 7.0) at a concentration of 10 mM, thus preparing a substrate solution. Next, 50 μl of each sample was added to a microplate, and 100 μl of the α-glucosidase enzyme solution was added, followed by incubation at 25° C. for 5 minutes. Next, 50 μl of the substrate solution was added, and after 2 minutes, the absorbance at 405 nm was measured with the Multi Detection Reader (Infinite 200, TECAN Group Ltd, Switzearland).

Inhibitory activity (%)=(absorbance of control)−(absorbance of sample-treated group)/(absorbance of control)×100

All experiments were repeated three times or more, and experimental results were calculated as mean±standard deviation (mean±S.D.) using the statistical program (SPSS ver. 12.0, SPSS Inc., Chicago, IL, USA). Statistically significant differences between experimental groups were tested using a one-way analysis of variance test (ANOVA), and in case of significance (p<0.05), a post-test was performed with the Duncan's multiple range test.

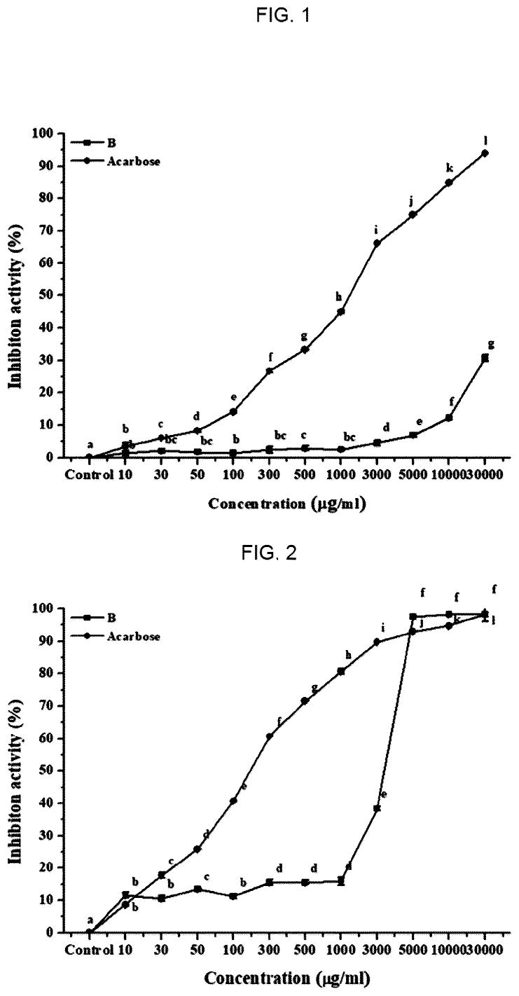

As a result of measuring and analyzing the α-glucosidase inhibitory activity of the beet mixture of Example 1, it was confirmed that the beet mixture exhibited α-glucosidase inhibitory activities of 1.28±0.60% at a concentration of 10 μg/ml, 2.00±0.70% at 30 μg/ml, 1.69±0.52% at 50 μg/ml, 1.37±0.09% at 100 μg/ml, 2.33±0.99% at 300 μg/ml, 2.77±0.80% at 500 μg/ml, 2.44±00.28% at 1 mg/ml, 4.45±0.79% at 3 mg/ml, 6.88±0.70% at 5 mg/ml, 12.14±0.35% at 10 mg/ml, and 30.58±1.01% at 30 mg/ml, which is the highest concentration of the sample, indicating that the beet mixture inhibited α-amylase activity (FIG. 1).

<1-2> Alpha-Amylase Assay

As an enzyme, porcine pancreatic alpha-amylase (Sigma, USA) was used at a concentration of 1 unit/ml, and as a substrate, 1% starch (Sigma, USA) was used. 100 μl of the sample, 250 μl of 1% starch, and 50 μl of alpha-amylase were used, and 48 mM DNS reagent was used as a detection reagent. The absorbance at 540 nm was measured by ELISA.

All experiments were repeated three times or more, and experimental results were calculated as mean±standard deviation (mean±S.D.) using the statistical program (SPSS ver. 12.0, SPSS Inc., Chicago, IL, USA). Statistically significant differences between experimental groups were tested using a one-way analysis of variance test (ANOVA), and in case of significance (p<0.05), a post-test was performed with the Duncan's multiple range test.

As a result of measuring and analyzing the α-amylase inhibitory activity of the beet mixture of Example 1, it was confirmed that the beet mixture exhibited α-amylase inhibitory activities of 11.51±0.83% at a concentration of 10 μg/ml, 10.55±0.82% at a concentration of 30 μg/ml, 13.36±0.41% at a concentration of 50 μg/ml, 11.19±0.81% at a concentration of 100 μg/ml, 15.44±0.79% at a concentration of 300 μg/ml, 15.46±0.65% at a concentration of 500 μg/ml, 15.97±1.21% at a concentration of 1 mg/ml, 38.36±0.61% at a concentration of 3 mg/ml, 97.57±0.06% at a concentration of 5 mg/ml, 98.19±0.12% at a concentration of 10 mg/ml, and 98.23±2.08% at a concentration of 30 mg/ml, and exhibited a higher α-amylase inhibitory activity than a negative control (acarbose) at a concentration of 5 mg/ml or higher (FIG. 2).

### <Experimental Example 2> SD Rat Animal Experiment

<2-1> Laboratory Animals and Breeding Environment

As experimental animals, 4-week-old specific-pathogen free (SPF) male SD rats were obtained from Samtako Korea (Osan, Korea), and acclimated for 1 week before use in the experiment. As an experimental food, normal mouse feed (Purina Lab Rodent Chow #38057, Purina Co., Seoul Korea) was fed, and during the acclimatization period, filtered water was replaced with fresh one and the animals were allowed access ad libitum to water. During the breeding period, the animals were kept under the following conditions: temperature, 23±1° C.; humidity, 50±5%; noise, 60 phone or less; lighting time, 08:00 to 20:00 (12 hours a day); illumination, 150 to 300 Lux; 10 to 12 ventilations per hour. This experiment was conducted in compliance with the Animal Experimental Ethics Regulations.

<2-2> Experimental Group Setting and Sample Administration

After completion of the acclimatization period, the experimental animals were divided into groups using the randomized block design based on fasting blood glucose that the mean values between the groups were uniform, and then the animals were marked using an ear punch. The animals were divided into a normal group, a control group, a group to which 10 mg/kg of the beet mixture was administered (10 mg/kg Beta vulgaris mixture group), a group to which 30 mg/kg of the beet mixture was administered (30 mg/kg Beta vulgaris mixture group), a group to which 30 mg/kg of the beet mixture was administered (30 mg/kg Beta vulgaris mixture group), and a positive control group (3 mg/kg acarbose), each consisting of 8 animals. Using these groups, an oral sucrose tolerance test (OSTT) and an oral starch tolerance test (OATT) were conducted.

<2-3> Test Method

Fasting blood glucose level was measured from the caudal veins of the experimental animals fasted for more than 8 hours, and then each sample was administered orally to each group. After 30 minutes, blood glucose was measured again, and 2 g/kg of glucose was administered to each groups. From this time point, blood glucose was measured every 30 minutes until 120 minutes. Changes in blood glucose levels in each group were analyzed based on the measured blood glucose levels.

All experimental results were calculated as mean±standard error (mean±S.E.) using the statistical program (SPSS ver. 12.0, SPSS Inc., Chicago, IL, USA). Statistically significant differences between experimental groups were tested using a one-way analysis of variance test (ANOVA), and in case of significance (p<0.05), a post-test was performed with the Duncan's multiple range test.

<2-4> Oral Starch Tolerance Test (OATT)

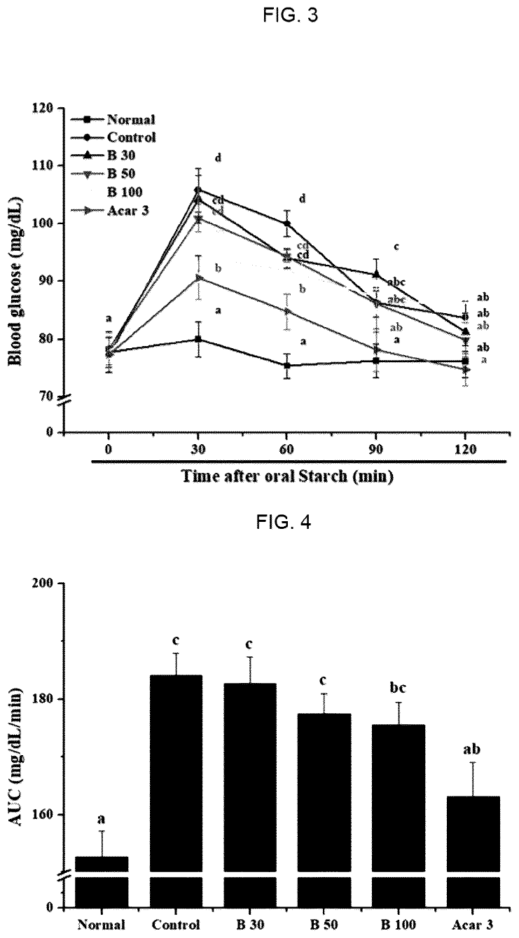

In order to evaluate the antidiabetic effect of the beet mixture of Example 1 at different concentrations, an oral starch tolerance test was conducted. As a result, it was shown that the blood glucose levels at 30 min after starch administration were 105.88±3.78 mg/dL in the control group, which was significantly higher than 80.00±3.06 mg/dL in the normal group, but 104.25±4.16 mg/dL in the 30 mg/kg beet mixture group (B 30), 101.10±2.41 mg/dL in the 50 mg/kg beet mixture group (B 50), 94.38±2.54 mg/dL in the 100 mg/kg beet mixture group (B 100), and 90.63±3.80 mg/dL in the positive control group (3 mg/kg acarbose, A 3), and the 50 mg/kg (B 50) and 100 mg/kg beet mixture (B 100)-administered groups exhibited a concentration-dependent decrease in blood glucose level compared to the control group (FIG. 3).

The above oral starch tolerance test results (FIG. 3) showed a tendency similar to the results of analysis of the area under the curve (AUC). The change in blood glucose after starch administration was 270.09±5.75 mg/dL in the control group, which was significantly higher than 135.67±5.81 mg/dL in the normal group, and were 264.79±6.37 mg/dL in the 30 mg/kg beet mixture group (B 30), 252.60±6.58 mg/dL in the 50 mg/kg beet mixture group (B 50), 246.02±5.55 mg/dL in the 100 mg/kg beet mixture group (B 100), and 197.32±7.12 mg/dL in the positive control group (3 mg/kg acarbose, A 3), and the changes in blood glucose were low in the order of the positive control group (A 3), the 100 mg/kg beet mixture group (B 100), the 500 mg/kg beet mixture group (B 50), and the 30 mg/kg beet mixture group (B 30) (FIG. 4).

<2-5> Oral Sucrose Tolerance Test (OSTT)

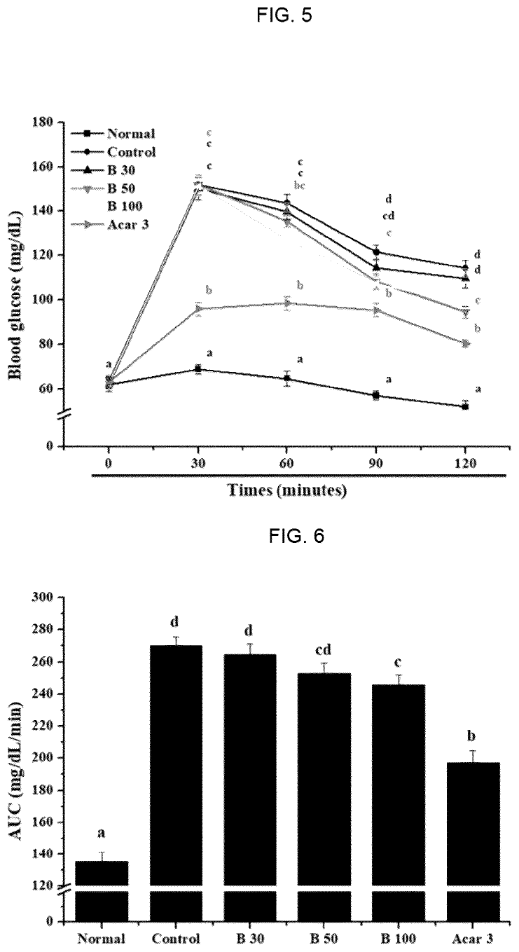

In order to further evaluate the antidiabetic effect of the beet mixture of Example 1 at concentrations, an oral sucrose tolerance test was conducted. As a result of the test, it was shown that the blood glucose levels at 30 min after sucrose administration were 152.00±4.80 mg/dL in the control group, which was significantly higher than 68.75±2.25 mg/dL in the normal group, and were 150.13±5.08 mg/dL in the 30 mg/kg beet mixture group (B 30), 152.13±4.64 mg/dL in the 50 mg/kg beet mixture group (B 50), 151.38±4.59 mg/dL in the 100 mg/kg beet mixture group (B 100), and 95.88±3.20 mg/dL in the negative control group (3 mg/kg acarbose, A 3). The difference in blood glucose level between the control group and the beet mixture-administered group started to appear from 60 min after sucrose administration, and the blood glucose levels at 120 min after sucrose administration were 114.2.98±2.98 mg/dL in the control group, 109.63±4.20 mg/dL in the 30 mg/kg beet mixture group (B 30), 94.50±2.77 mg/dL in the 50 mg/kg beet mixture group (B 50), 97.50±3.33 mg/dL in the 100 mg/kg beet mixture group (B 100), and 80.38±1.76 mg/dL in the positive control group (3 mg/kg acarbose, A 3), and thus the blood glucose levels at 120 min after sucrose administration were low in the order of the positive control group (3 mg/kg acarbose, A 3), the 50 mg/kg beet mixture group (B 50), the 100 mg/kg beet mixture group (B 100), and the 50 mg/kg beet mixture group (B 50) (FIG. 5).

The above oral sucrose tolerance test results (FIG. 5) showed a tendency similar to the results of analysis of the area under the curve (AUC). The changes in blood glucose level after sucrose administration were 270.09±5.75 in the control group, which was significantly higher than 135.67±5.81 mg/dL in the normal group, but 264.79±6.37 mg/dL in the 30 mg/kg beet mixture group (B 30), 252.60±6.58 mg/dL in the 50 mg/kg beet mixture group (B 50), 246.02±5.55 mg/dL in 100 mg/kg beet mixture group (B 100), and 197.32±7.12 mg/dL in the positive control group (3 mg/kg acarbose, A 3), indicating that administration of the beet mixture reduced the blood glucose level in a concentration-dependent level, and the blood glucose level was significantly lower in the 100 mg/kg beet mixture group (B 100) than in the control group.

Based on these results, 50 mg/kg of the beet mixture was selected as the highest concentration using an extrapolation formula (rat: 0.081, mouse: 0.162) to reflect it in future type 2 diabetes animal experiments, and used in subsequence type 2 diabetes animal experiments with db/db mice.

### <Experimental Example 3> db/db Mouse Animal Experiment

<3-1> Laboratory Animals and Breeding Environment

As experimental animals, 5-week-old specific-pathogen free (SPF) male db/db mice were obtained from Central Laboratory Animal, Inc. (Seoul, Korea), and acclimated for 1 week before use in the experiment. As an experimental food, normal mouse feed (Purina Lab Rodent Chow #38057, Purina Co., Seoul Korea) was fed, and during the acclimatization period, filtered water was replaced with fresh one daily and the animals were allowed access ad libitum to water. During the breeding period, the animals were kept under the following conditions: temperature, 23±1° C.; humidity, 50±5%; noise, 60 phone or less; lighting time, 08:00 to 20:00 (12 hours a day); illumination, 150 to 300 Lux; 10 to 12 ventilations per hour. This experiment was conducted in compliance with the Animal Experimental Ethics Regulations.

<3-2> Experimental Group Setting and Sample Administration

After completion of the acclimatization period, the experimental animals were divided into groups using the randomized block design based on fasting blood glucose that the mean values between the groups were uniform, and then the animals were marked using an ear punch. The animals were divided into a normal group, a control group, a group to which 10 mg/kg of the beet mixture was administered (10 mg/kg Beta vulgaris mixture group), a group to which 30 mg/kg of the beet mixture was administered (30 mg/kg Beta vulgaris mixture group), a group to which 30 mg/kg of the beet mixture was administered (30 mg/kg Beta vulgaris mixture group), and a positive control group (300 mg/kg metformin), each consisting of 8 animals. Each sample was orally administered 5 times a week for 3 weeks.

<3-3> Test Method

Weekly body weight and blood sugar level were all measured once a week at a certain time, and food intake and water intake were measured the next day after feeding certain amounts of food and water once a week. Weekly blood glucose levels were measured using blood collected from the caudal veins using a blood glucose meter (AutoCheck, Diatech Korea) at a certain time every week. For autopsy, blood was collected from the abdominal vena cava after inhalation anesthesia and used for blood analysis, and the liver, epididymal fat, pancreas, and kidney were harvested and weighed.

Blood was collected from the abdominal vena cava, placed in conical tubes, coagulated for 30 minutes at room temperature, and then centrifuged at 3,000 rpm for 10 minutes. Analysis of blood fructosamine, TC, TG, LDL-C, HDL-C, and VLDL levels were performed by KPNT (Cheongju, Korea), and insulin analysis was performed using an ELISA kit (Crystalchem, USA). HOMA-IR and HOMA-beta levels were calculated based on the blood analysis results.

Analysis of the islets of Langerhans in the pancreatic tissue was performed by immunostaining. The fixed tissue was embedded in paraffin, sectioned at a thickness of 4 μm, and the deparaffinized tissue was bathed for 20 min at 60° C. with an antigen retriever (Vector, USA). After cooling for 20 min, the sections were treated with BLOXALL™ Blocking Solution (Vector, USA) for 10 min. After washing twice with TBST buffer for 5 min, samples were reacted with 2.5% normal goat serum for blocking (Vector, USA) for 20 min, and then the blocking reagent and working insulin antibody (Cell Signaling, MA, USA) solution (2 to 10 μl of antibody to 1 ml of Ready-To-Use 2.5% normal goat serum for blocking) was reacted at 4° C. overnight. Next, the tissue was reacted for 1 hour at room temperature using an ImmPRESS™ horseradish peroxidase anti-rabbit IgG (peroxidase) polymer detection kit (Vector, USA), and then washed twice with TBST buffer for 5 min, and stained using chromogen AEC (Vector, USA) and hematoxylin. The stained pancreatic tissue was observed and photographed using an optical microscope (Olympus BX50 F4; Olympus, Tokyo, Japan).

All experimental results were calculated as mean±standard error (S.E.) using SPSS ver. 12.0 (SPSS Inc., Chicago, IL, USA). Statistically significant differences between experimental groups were tested using a one-way analysis of variance test (ANOVA), and in case of significance (p<0.05), a post-test was performed with the Duncan's multiple range test.

<3-4> Weekly Biomarker Analysis

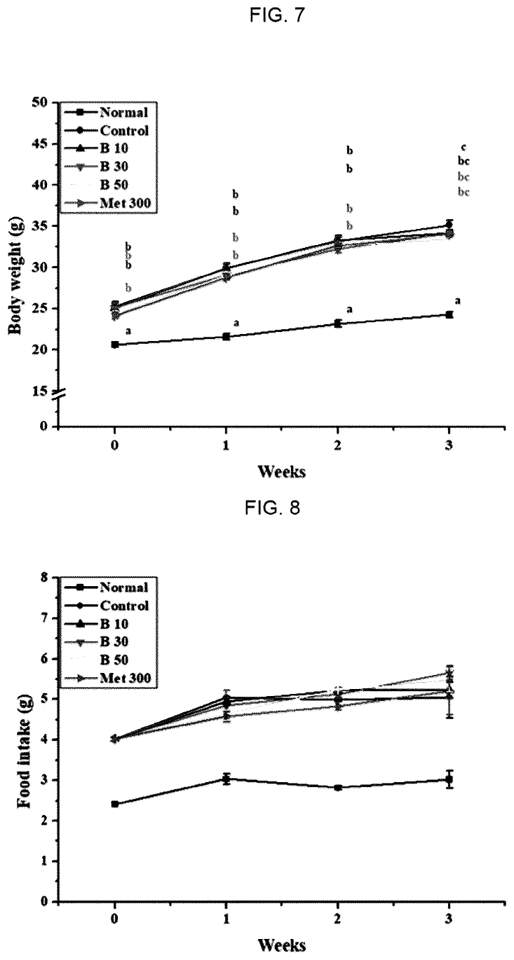

In order to evaluate the effect of the beet mixture of Example 1 on the weekly body weight change in the type 2 diabetes model, the body weight of each mouse was measured once a week. As a result, the body weights of the experimental groups at week 3 (the time of the end of the test) were 24.26±0.35 g in the normal group, 35.13±0.57 g in the control group, 34.16±0.41 g in the 10 mg/kg beet mixture group (B 10), 34.10±0.29 g in the 30 mg/kg beet mixture group (B 30), 33.28±0.34 g in the 50 mg/kg beet mixture group (B 50), and 34.04±0.35 g in the positive control group (300 mg/kg metformin, Met 300), indicating that the body weight of the control group was the highest. On the other hand, the body weight of each of the beet mixture groups (30 mg/kg and 50 mg/kg) and the positive control group (300 mg/kg metformin, Met 300) was lower than that of the control group, and in particular, the 50 mg/kg beet mixture group had a significant difference in body weight from the control group (FIG. 7).

In addition, after administration of the beet mixture (10 mg/kg, 30 mg/kg, and 50 mg/kg), changes in weekly food intake and water intake were analyzed.

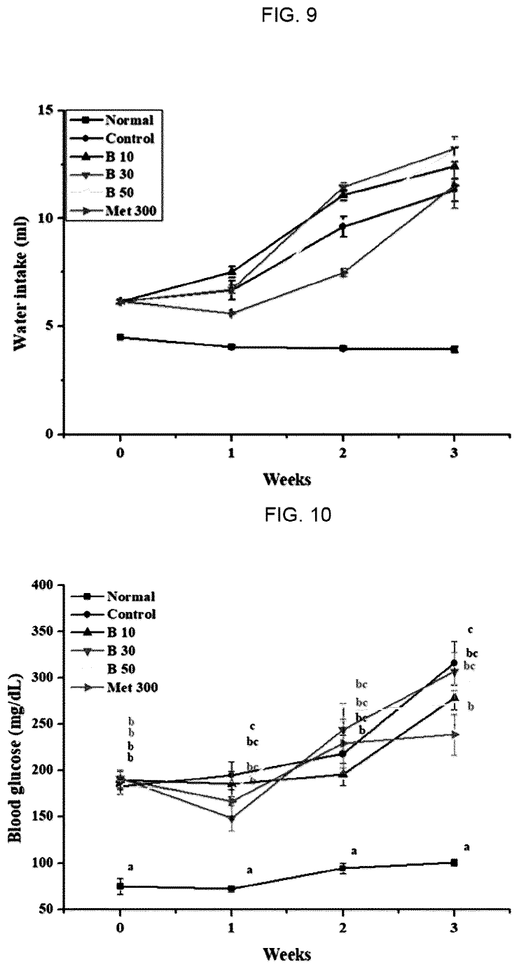

As a result of measuring the weekly food intake and water intake for 3 weeks, it was shown that the weekly food intake and water intake were higher in the experimental groups than in the normal group, but there was no significant difference between the experimental groups (FIGS. 8 and 9).

In addition, the effect of the beet mixture on the weekly blood glucose change after 3 weeks of oral administration in the type 2 diabetes model was evaluated.

It was observed that the blood glucose levels at 1 week after sample administration were 194.9±15.0 mg/dL in the control group, which was significantly higher than 72.3±3.6 mg/dL in the normal group, and were 185.9±13.6 mg/dL in the 10 mg/kg beet mixture group (B 10), 148.5±14.0 mg/dL in the 30 mg/kg beet mixture group (B 30), 179.1±9.3 mg/dL in the 50 mg/kg beet mixture group (B 50), and 167.0±19.1 mg/dL in the positive control group (300 mg/kg metformin, Met 300), indicating that the blood glucose levels were low in the order of the 10 mg/kg beet mixture group (B 10), the 50 mg/kg beet mixture group (B 50), the positive control group (300 mg/kg metformin, Met 300), and the 30 mg/kg beet mixture group (B 30). In addition, it was observed that the blood glucose levels at 2 weeks after sample administration were low in the order of the 10 mg/kg beet mixture group (B 10), the control group, the positive control group (300 mg/kg metformin, Met 300), the 30 mg/kg beet mixture group (B 30), and the 50 mg/kg beet mixture group (B 50). However, it was observed that the blood glucose levels in the experimental groups started to decrease compared to that of the control group from 3 weeks after sample administration, and the blood glucose levels in the groups were 100.4±3.7 mg/dL in the normal group, 316.0±23.6 mg/dL in the control group, 278.9±13.0 mg/dL in the 10 mg/kg beet mixture group (B 10), 307.1±20.6 mg/dL in the 30 mg/kg beet mixture group (B 30), 271.5±22.0 mg/dL in the 50 mg/kg beet mixture group (B 50), and 238.8±21.9 mg/dL in the positive control group (300 mg/kg metformin, Met 300), indicating that the blood glucose level in the control group was the highest. On the other hand, it was observed that the blood glucose levels in the groups to which different concentrations of the beet mixture (10 mg/kg, B 10, 30 mg/kg, B 30, and 50 mg/kg, B 50) were administered were lower than that in the control group in a concentration-dependent manner (FIG. 10).

<3-5> Oral Glucose Tolerance Test

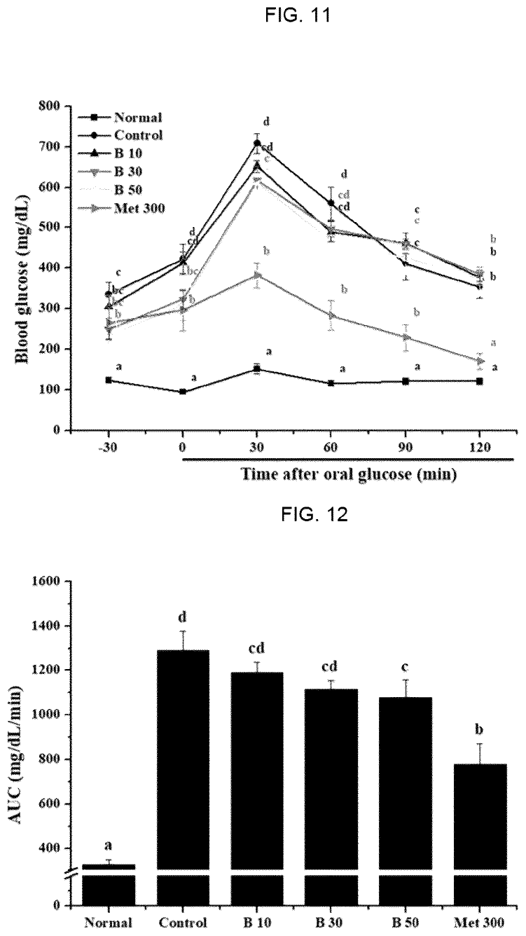

To evaluate the antidiabetic effect of the beet mixture, an oral glucose tolerance test was conducted. As a result of the test, it was shown that the blood glucose levels at 30 min after oral glucose administration were 708.6±24.6 mg/dL in the control group, which was significantly higher than 151.0±12.9 mg/dL in the normal group, and were 651.8±14.7 mg/dL in the 10 mg/kg beet mixture group (B 10), which was lower than that in the control group. On the other hand, it was observed that the blood glucose levels at 30 min after oral glucose administration were 617.3±34.2 mg/dL in the 30 mg/kg beet mixture group (B 30), 608.8±25.1 mg/dL in the 50 mg/kg beet mixture group (B 50), and 382.0±31.0 mg/dL in the positive control group (300 mg/kg metformin, Met 300), which were significantly different from that in the control group, and the blood glucose levels were low in the order of the positive control group (300 mg/kg metformin, Met 300), the 50 mg/kg beet mixture group (B 50), the 30 mg/kg beet mixture group (B 30), and the 10 mg/kg beet mixture group (B 10) (FIG. 11).

<3-6> Analysis of Oral Glucose Tolerance Test (OGTT) Results

The changes in blood glucose level after glucose administration were 1291.77±244.59 mg/dL in the control group, which was significantly higher than 327.03±60.41 mg/dL in the normal group, and were 1191.85±133.21 mg/dL in the 10 mg/kg beet mixture group (B 10), and 1114.08±117.02 mg/dL in the 30 mg/kg beet mixture group (B 30), which were lower than that in the control group. On the other hand, it was observed that the changes in blood glucose level were 1077.63±233.15 mg/dL in the 50 mg/kg beet mixture group (B 50), and 778.06±262.53 mg/dL in the positive control group (300 mg/kg metformin, Met 300), which were significantly lower than that in the control group (FIG. 12).

<3-7> Analysis of Glycated Hemoglobin (HbA1c)

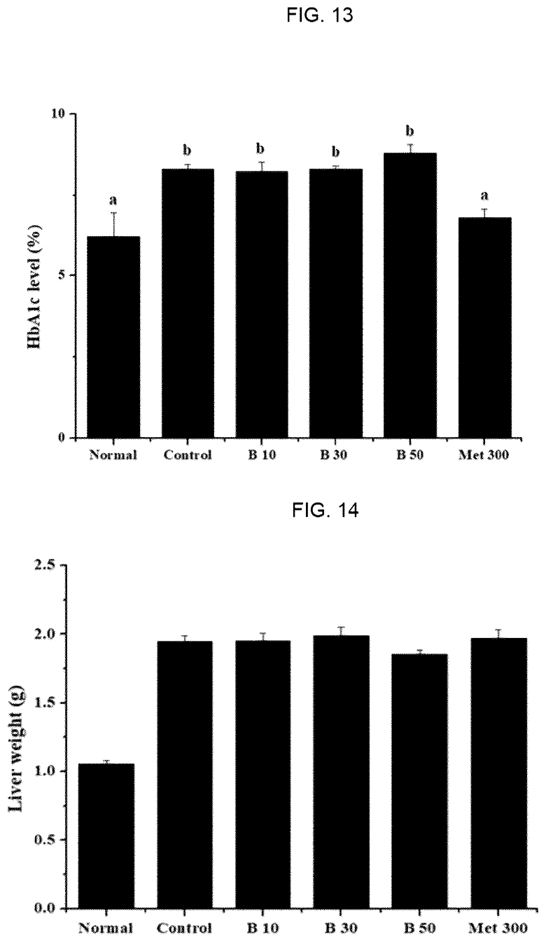

To confirm the change in blood sugar level after 3 weeks of administration of the beet mixture, glycated hemoglobin (HbA1c) was comparatively analyzed. As a result of analysis, it was shown that the HbA1c level was 8.30±0.14% in the control group, which was significantly higher than 6.21±0.74% in the normal group. In addition, it was observed that the HbA1c levels were 6.79±0.29% in the positive control group (300 mg/kg metformin, Met 300), which was similar to that in the normal group, but were 8.23±0.30% in the 10 mg/kg beet mixture group (B 10), 8.29±0.12% in the 30 mg/kg beet mixture group (B 30), and 8.80±0.25% in the 50 mg/kg beet mixture group (B 50), which were not significantly different from that in the control group (FIG. 13).

<3-8> Analysis of Tissue Weight

The effects of the beet mixture on the weights of liver, kidney and epididymal tissues in the type 2 diabetes model were measured after 3 weeks of administration of the beet mixture. First, it was observed that the liver tissue weights in the groups were 1.05±0.0. g in the normal group, 1.95±0.04 g in the control group, 1.95±0.05 g in the 10 mg/kg beet mixture group (B 10), 1.99±0.06 g in the 30 mg/kg beet mixture group (B 30), 1.85±0.03 g in the 50 mg/kg beet mixture group (B 50), and 1.97±0.06 g in the positive control group (300 mg/kg metformin, Met 300), which were significantly different from that in the control group (FIG. 14).

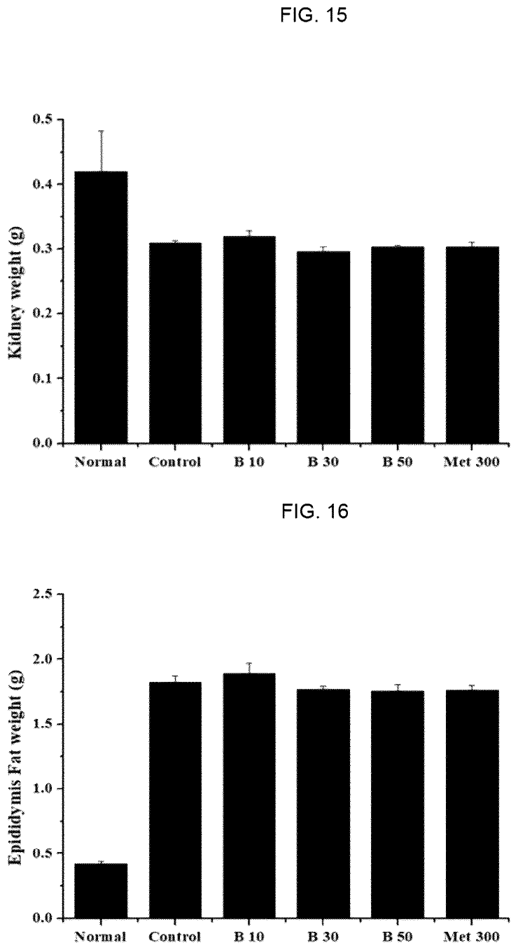

It was shown that the kidney tissue weights were 0.420±0.063 g in the normal group, 0.309±0.004 g in the control group, 0.319±0.010 g in the 10 mg/kg beet mixture group (B 10), 0.296±0.007 g in the 30 mg/kg beet mixture group (B 30), 0.303±0.003 g in the 50 mg/kg beet mixture group (B 50), and 0.303±0.008 g in the positive control group (300 mg/kg metformin, Met 300), indicating that there was no significant difference in kidney tissue weight between the groups (FIG. 15).

In addition, as a result of measuring the weight of epididymal fat tissue at autopsy, the weight of epididymal fat tissue was 1.823±0.050 in the control group, which was significantly higher than 0.420±0.022 g in the normal group. In addition, the epididymal fat tissue weights in the other groups were 1.894±0.078 g in the 10 mg/kg beet mixture group (B 10), 1.770±0.024 g in the 30 mg/kg beet mixture group (B 30), 1.755±0.038 g in the 50 mg/kg beet mixture group (B 50), and 1.761±0.038 g in the negative control group (300 mg/kg metformin, Met 300), which were not significantly different from that in the control group (FIG. 16).

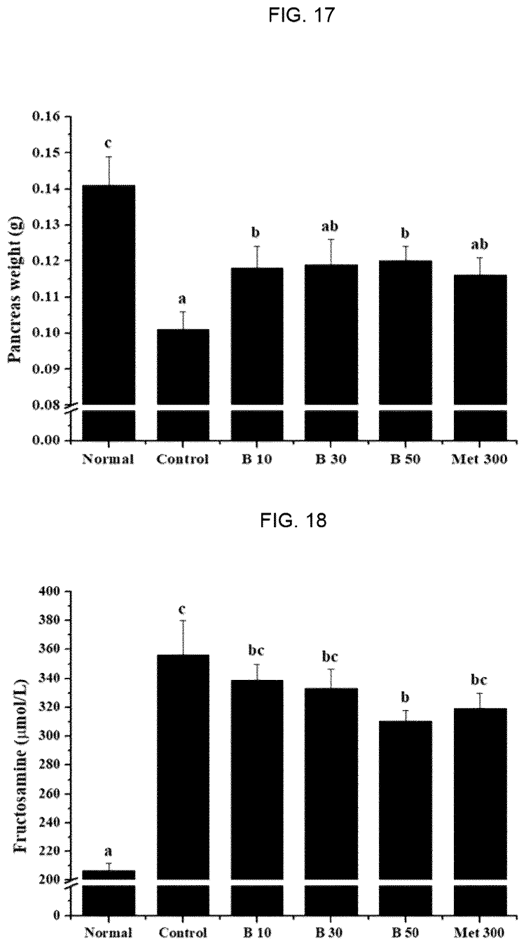

In addition, to evaluate the effect of the beet mixture on the pancreatic tissue weight in the type 2 diabetes model, the pancreatic tissue weight was measured at autopsy. It was observed that the pancreatic tissue weight was 0.101±0.005 g in the control group, which was significantly lower than 0.141±0.008 g in the normal group. On the other hand, the pancreatic tissue weights in the groups to which each of different concentrations of the beet mixture and the positive control (100 mg/kg metformin) were 0.118±0.006 g in the 10 mg/kg beet mixture group (B 10), 0.119±0.007 g in the 30 mg/kg beet mixture group (B 30), 0.120±0.004 g in the 50 mg/kg beet mixture group (B 50), and 0.116±0.005 g in the positive control group (300 mg/kg metformin, Met 300), indicating that the pancreatic tissue weight in the 30 mg/kg beet mixture group (B 30) and the positive control group (300 mg/kg metformin, Met 300) tended to increase compared to that in the control group. In particular, it was observed that the pancreatic tissue weight in the 10 mg/kg beet mixture group (B 10) and the 50 mg/kg beet mixture group (B 50) significantly increased compared to that in the control group (FIG. 17).

<3-9> Hematological Analysis

As a result of analyzing the effect of the beet mixture on serum fructosamine level, the fructosamine level was 356.50±23.71 μmol/L in the control group, which was significantly higher than 206.50±5.04 μmol/L in the normal group. The fructosamine levels in the groups to which different concentrations of the beet mixture were administered were 356.50±10.78 μmol/L in the 10 mg/kg beet mixture group (B 10), and 333.25±12.89 μmol/L in the 30 mg/kg beet mixture group (B 30), which were lower than that in the control group. On the other hand, the 50 mg/kg beet mixture group (B 50) showed a serum fructosamine level of 310.75±7.07 μmol/L, which was significantly lower than that in the control group. The positive control group (300 mg/kg metformin, Met 300) showed a serum fructosamine level of 319.13±10.89 μmol/L, and the serum fructosamine level was low in the order of the 50 mg/kg beet mixture group (B 50), the positive control group (300 mg/kg metformin, Met 300), the 30 mg/kg beet mixture group (B 30), and the 10 mg/kg beet mixture group (B 10) (FIG. 18).

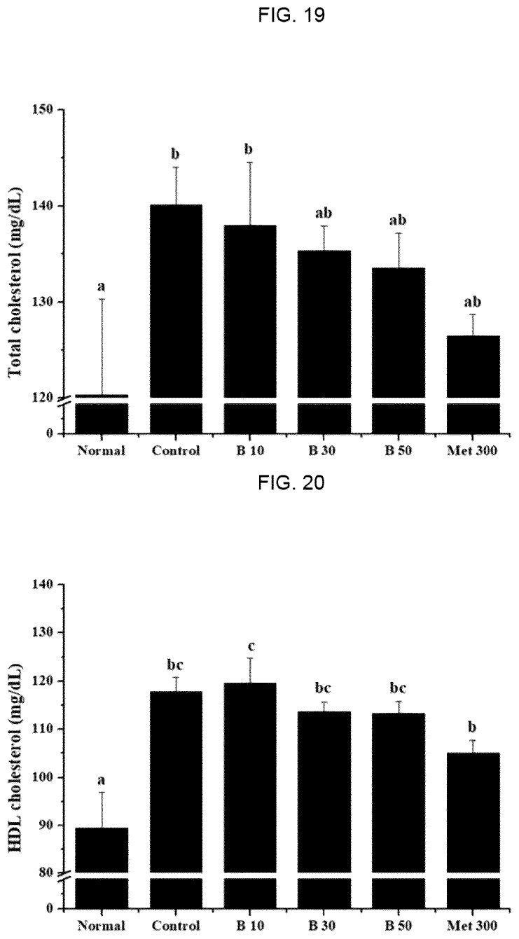

As a result of analyzing the serum total cholesterol level in each group, it was shown that the total cholesterol levels were 120.38±9.91 mg/dL in the normal group, 140.13±3.88 mg/dL in the control group, 138.00±6.52 mg/dL in the 10 mg/kg beet mixture group (B 10), 135.38±2.52 mg/dL in the 30 mg/kg beet mixture group (B 30), 133.50±3.68 mg/dL in the 50 mg/kg beet mixture group (B 50), and 126.50±2.24 mg/dL in the positive control group (300 mg/kg metformin, Met 300), indicating that the total cholesterol levels in all the experimental groups except for the 10 mg/kg beet mixture group (B 10) tended to decrease compared to that in the control group (FIG. 19).

As a result of analyzing the effect of the beet mixture on serum HDL cholesterol level, it was shown that the HDL cholesterol level was 117.83±2.92 mg/dL in the control group, which was significantly higher than 89.58±7.33 in the normal group. On the other hand, the 10 mg/kg beet mixture group (B 10) showed an HDL cholesterol level of 119.69±2.92 mg/dL, which was similar to but higher than that in the control group. In addition, the HDL cholesterol levels in the other groups were 113.70±1.99 mg/dL in the 30 mg/kg beet mixture group (B 30), 113.25±2.57 mg/dL in the 50 mg/kg beet mixture group (B 50), and 105.09±2.63 mg/dL in the positive control group (300 mg/kg metformin, Met 300), which were lower than that in the control group, indicating that the HDL cholesterol levels in all the experimental groups except for the 10 mg/kg beet mixture group (B 10) tended to decrease compared to that in the control group (FIG. 20).

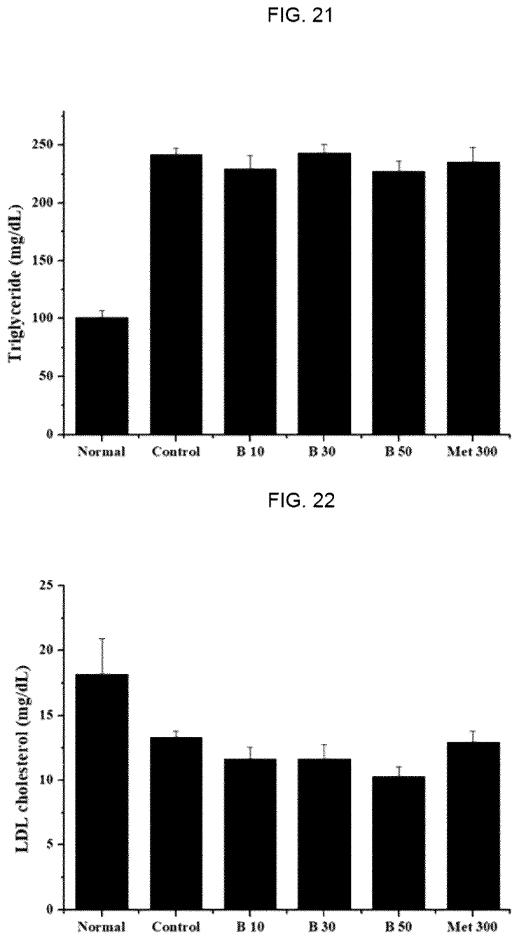

The effects of the beet mixture on serum triglyceride and LDL cholesterol levels were analyzed after 3 weeks of administration of the beet mixture. First, as a result of analyzing the effect of the beet mixture on serum triglyceride levels, it was shown that the serum triglyceride levels were 242.38±5.33 mg/dL in the control group, which was significantly higher than 101.25±5.80 mg/dL in the control group, and were 229.63±11.52 mg/dL in the 10 mg/kg beet mixture group (B 10), 243.50±7.30 mg/dL in the 30 mg/kg beet mixture group (B 30), 227.50±9.24 mg/dL in the 50 mg/kg beet mixture group (B 50), and 236.13±11.87 mg/dL in the positive control group (300 mg/kg metformin, Met 300), which were not significantly different from that in the control group (FIG. 21).

As a result of measuring LDL cholesterol levels, it was shown that the LDL cholesterol levels were 13.29±0.49 mg/dL in the control group, which was lower than 18.14±2.81 mg/dL in the normal group, and were 11.65±0.94 mg/dL in the 10 mg/kg beet mixture group (B 10), 11.68±1.10 mg/dL in the 30 mg/kg beet mixture group (B 30), 10.29±0.73 mg/dL in the 50 mg/kg beet mixture group (B 50), and 12.94±0.84 mg/dL in the positive control group (300 mg/kg metformin, Met 300), indicating that the LDL cholesterol levels were not significantly different between the experimental groups (FIG. 22).

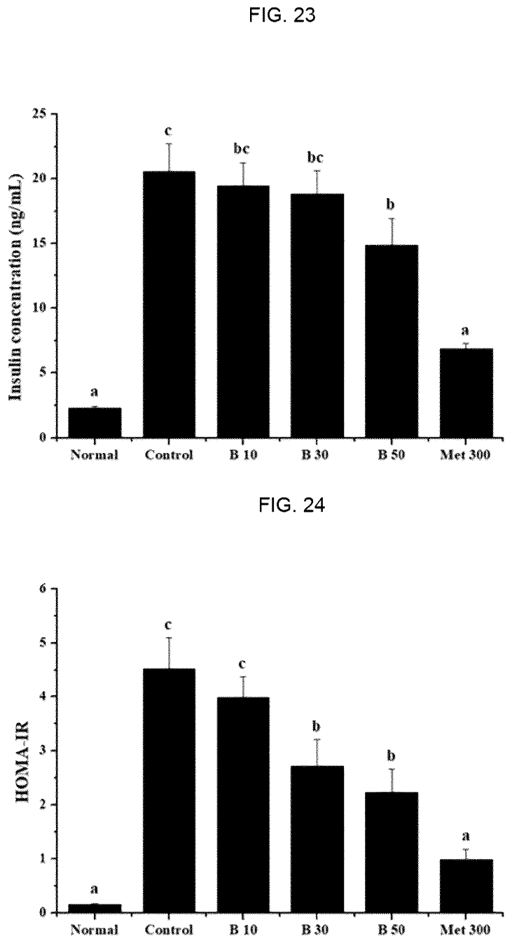

After 3 weeks of administration of the beet mixture, blood was collected from each experimental group, serum was separated therefrom, and the serum insulin level in each experimental group was measured. As a result, it was shown that the serum insulin level in the normal group was 2.305±0.105 ng/mL, which was significantly lower than 20.545±4.528 ng/mL in the control group. In addition, it was shown that the serum insulin levels in the groups to which the beet mixture was added were 19.469±1.767 ng/mL in the 10 mg/kg beet mixture group (B 10), and 18.850±1.796 ng/mL in the 30 mg/kg beet mixture group (B 30), which were lower than that in the control group, and the serum insulin levels were 19.469±2.076 ng/mL in the 50 mg/kg beet mixture group (B 50), and 12.94±0.84 ng/mL in the positive control group (300 mg/kg metformin, Met 300), which were significantly lower than that in the control group, indicating that the serum insulin levels were low in the order of the positive control group (300 mg/kg metformin, Met 300), the 50 mg/kg beet mixture group (B 50), the 30 mg/kg beet mixture group (B 30), and the 10 mg/kg beet mixture group (B 10) (FIG. 23).

Using the above-described results of measuring serum insulin levels and blood glucose levels, HOMA-IR (homeostasis model assessment for insulin resistance, insulin resistance) and HOMA-β (homeostasis model assessment of p-cell function, insulin secretion ability) were analyzed. As a result of analysis, the HOMA-IR levels in the groups were 4.528±0.570 in the control group, which was significantly higher than 0.153±0.012 in the normal group, and 3.993±0.391 in the 10 mg/kg beet mixture group (B 10), which was lower than that in the control group. In addition, it was shown that the HOMA-IR levels were 2.718±0.484 in the 30 mg/kg beet mixture group (B 30), and 2.234±0.430 in the 50 mg/kg beet mixture group (B 50), which were significantly lower than that in the control group, indicating that the HOMA-IR level decreased in a concentration-dependent manner. The HOMA-IR level in the positive control group (300 mg/kg metformin, Met 300) was 0.975±0.199 which was similar to that in the normal group. Thus, the HOMA-IR level (insulin resistance) was low in the order of the positive control group (300 mg/kg metformin, Met 300), the 50 mg/kg beet mixture group (B 50), the 30 mg/kg beet mixture group B 30), and the 10 mg/kg beet mixture group (B 10) (FIG. 24).

In addition, the HOMA-β level in the normal group was 4.211±0.743, which was the lowest among the groups, and the HOMA-β level in the control group was 20.545±2.174, which was the highest among the groups. The HOMA-β levels in the experimental groups to which the beet mixture was administered were 19A69±1.767 in the 10 mg/kg beet mixture group (B 10), and 18.850±1.796 in the 30 mg/kg beet mixture group (B 30), which were lower than that in the control group. On the other hand, it was shown that the HOMA-β level in the 50 mg/kg beet mixture group (B 50) was 18.850±1.796, which was significantly lower than that in the control group, and the HOMA-β level in the positive control group (300 mg/kg metformin, Met 300) was 6.863±0.404, which was similar to 4.211±0.743 in the normal group (FIG. 25).

<3-10> Histological Analysis

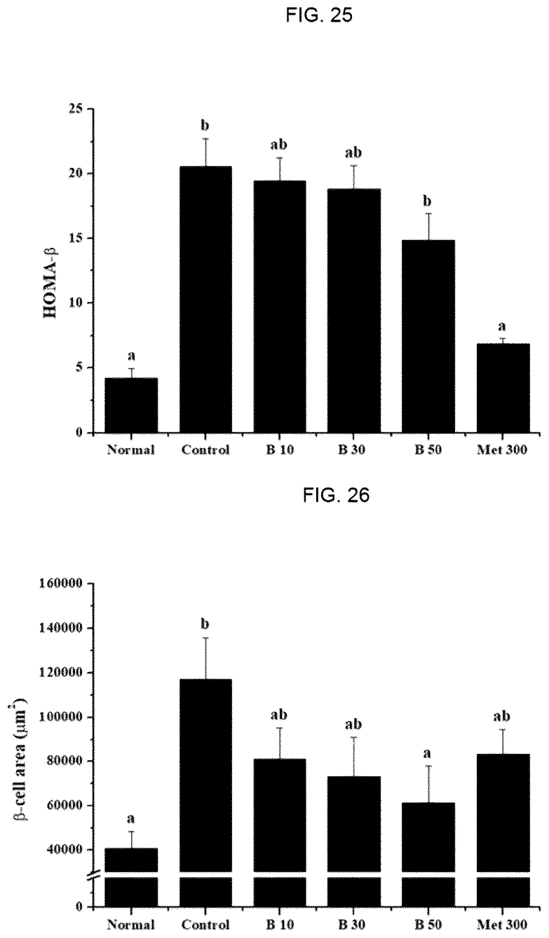

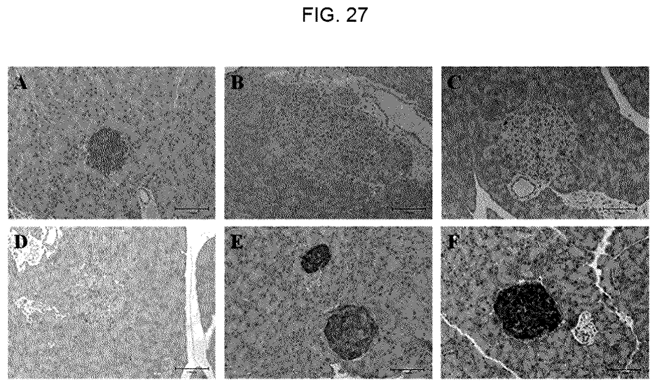

To evaluate the effect of the beet mixture on the p-cell area in the diabetes model, the p-cell area in each experimental group was analyzed using immunohistological staining. As a result of analysis, it was shown that the p-cell areas were 40679.75±7860A6 μm² in the normal group, 117047.02±18550.84 μm² in the control group, 81080.95±14114.83 μm² in the 10 mg/kg beet mixture group (B 10), 703402A6±17508.66 μm² in the 30 mg/kg beet mixture group (B 30), 61528.17±16512.57 μm² in the 50 mg/kg beet mixture group (B 50), and 83242.23±11320.56 μm² in the positive control group (300 mg/kg metformin, Met 300), indicating that the p-cell area decreased in all the experimental groups to which the beet mixture was administered, compared to the control group, and particularly, the β-cell area was significantly low in the 50 mg/kg beet mixture group (B 50) (FIGS. 26 and 27).

1. A food composition for preventing and alleviating diabetes, the food composition containing a fermentation broth obtained by juicing beets, carrots and apples at a weight ratio of 1.5 to 3.5:1.5 to 2.5:4 to 7, mixing the juices together, and fermenting the juice mixture at low temperature.

2. The food composition according to claim 1, wherein an acidity of the mixture is pH 4 to 5.5.

3. The food composition according to claim 1, wherein the fermenting at low temperature is performed at a low temperature of −3° C. to 10° C. for 1 to 3 days.

4. A food composition for preventing and alleviating diabetes, the food composition containing a fermentation broth obtained by low-temperature fermentation of a mixture of:
a first fermentation broth obtained by juicing beets, carrots and apples at a weight ratio of 1.5 to 3.5:1.5 to 2.5:4 to 7, mixing the juices together, and fermenting the juice mixture at low temperature; and
a second juice obtained by mixing tomatoes, broccoli, and cabbage at a weight ratio of 0.2 to 0.5:1 to 2:1 to 2, followed by boiling, and juicing the boiled mixture.

5. The food composition according to claim 4, wherein the first fermentation broth and the second juice are mixed together at a weight ratio of 1 to 2:1.

6. The food composition according to claim 4, wherein the mixture of the first fermentation broth and the second juice is fermented at a low temperature of −3° C. to 10° C. for 1 to 8 hours.

7. A method for preparing a food composition for preventing and alleviating diabetes, the method comprising steps of:
i) juicing beets, carrots and apples at a weight ratio of 1.5 to 3.5:1.5 to 2.5:4 to 7, and mixing the juices together, thereby preparing a mixture; and
ii) fermenting the mixture of step i) at a low temperature of −3° C. to 10° C. for 1 to 3 days, thereby preparing a fermentation broth.

8. The method of claim 7, further comprising steps of:
b) mixing tomatoes, broccoli and cabbage at a weight ratio of 0.2 to 0.5:1 to 2:1 to 2, followed by boiling, and juicing the boiled mixture, thereby preparing a juice;
c) mixing the fermentation broth of step a) and the juice of step b) at a weight ratio of 1 to 2:1, thereby preparing a mixture; and
d) fermenting the mixture of step c) at a low temperature of −3° C. to 10° C. for 1 to 8 hours.

9.-12. (canceled)
